(19) United States
(12) Patent Application Publication
(10) Pub. No.: US 2026/0345062 A1
(43) Pub. Date: Nov. 5, 2026

(54) BLOOD-BASED COGNITIVE ABILITY EVALUATION METHOD AND DEVICE

(71) Applicant: EWHA UNIVERSITY - INDUSTRY COLLABORATION FOUNDATION, Seoul (KR)

(72) Inventors: Young Mi PARK, Seoul (KR); Geon Ha KIM, Seoul (KR); Jang Hwan CHOI, Seoul (KR); Hyo Bin LEE, Seoul (KR)

(21) Appl. No.: 19/487,768

(22) PCT Filed: May 31, 2024
(86) PCT No.: PCT/KR2024/007480

§ 371 Date: Nov. 25, 2025

(30) Foreign Application Priority Data
May 31, 2023 (KR) 10-2023-0069953
Nov. 15, 2023 (KR) 10-2023-0158640

(51) Int. Cl. A61B 5/00; A61B 5/145

(52) U.S. Cl. CPC A61B 5/4088; A61B 5/14546; A61B 5/7271

(57) ABSTRACT

The present application relates to a blood-based cognitive ability evaluation method. The blood-based cognitive ability evaluation method includes: obtaining actual test data including actual blood test results and cognitive function test results of subjects; determining major cognitive function items of a cognitive function test related to whether the subjects belong to a first group, based on the actual test data; generating augmented test data, based on the obtained actual test data; predicting, by using a classification algorithm, whether pieces of individual data of the augmented test data belong to the first group; classifying the pieces of individual data of the augmented test data and the actual test data into one of the first group and a second group; and extracting one or more major blood factors associated with the first group for each of the major cognitive function items, based on the classified actual test data and the classified augmented test data.

### TECHNICAL FIELD

Embodiments of the present disclosure relate to a blood-based cognitive ability evaluation method and device.

The present application is the result of research conducted with the support of the Korea Health Industry Development Institute (No. HU20C0271) and the National IT Industry Promotion Agency (No. RS-2022-00155966, Artificial Intelligence Convergence Innovation Talent Development (Ewha Womans University), Project Identification Number: 1711179344) with funding from the government (Ministry of Health and Welfare and Ministry of Science and ICT) in 2024.

### BACKGROUND ART

As the aging population rapidly increases, health care for the elder is emerging as a major social concern, and in particular, research is actively being conducted on changes in cognitive function in old age in relation to brain health. Although cognitive function in old age is generally impaired or deteriorated with age, this change is not fixed and is an individual attribute which may vary depending on various factors.

Cognitive function tests, which measure cognitive function, are mainly used to diagnose diseases, such as mild cognitive impairment or dementia, and are conducted by computer programs using documents or based on documents. However, such document-based cognitive function tests are time-consuming and thus burdensome for the elderly people, and are difficult to use for the elderly people who have difficulty reading text. Furthermore, there was a problem in that it was impossible to perform a uniform follow-up observation on changes in cognitive function because the same test could not be conducted multiple times.

On the other hand, existing research on cognitive function has been mainly conducted on patients with diseases such as Alzheimer's disease, Parkinson's disease, diabetes, or hypertension, but recent research on super-agers, which refers to the elder with strong cognitive abilities, compared to their age, has been attracting attention. Super-agers have cognitive abilities significantly superior to their age, allowing them to live healthier lives physically and mentally. Thus, such super-agers may be used as a measure to evaluate the cognitive function of the elder.

Therefore, there is a growing need for the development of cognitive ability measurement technology which may be used by healthy subjects rather than for the purpose of diagnosing diseases, and there is a need for the development of cognitive ability evaluation technology which evaluates whether the subjects have strong cognitive abilities, compared to their age.

### DISCLOSURE OF INVENTION

### Technical Problem

Embodiments of the present disclosure are intended to solve the above-described problems and/or limitations, and aim to provide a cognitive ability evaluation method capable of evaluating cognitive ability, based on a blood test.

In addition, the present disclosure aims to provide a computer-readable recording medium having recorded thereon a program for causing a computer to perform the above-described method.

However, this is only an example and the scope of the present disclosure is not limited thereto.

### Solution to Problem

According to an embodiment of the present disclosure, a blood-based cognitive ability evaluation method may include: obtaining actual test data including actual blood test results and cognitive function test results of subjects; determining major cognitive function items of a cognitive function test related to whether the subjects belong to a first group, based on the actual test data; generating augmented test data, based on the obtained actual test data; predicting, by using a classification algorithm, whether pieces of individual data of the augmented test data belong to the first group; classifying the pieces of individual data of the augmented test data and the actual test data into one of the first group and a second group; and extracting one or more major blood factors associated with the first group for each of the major cognitive function items, based on the classified actual test data and the classified augmented test data.

In the present disclosure, the determining of the major items of the cognitive function test may include: classifying the respective subjects into the first group or the second group; training a correlation between each item of the cognitive function test and whether the subjects belong to the first group, based on the classification of the subjects into the first or second group and the actual test data; and determining the major cognitive function items, based on the training.

In the present disclosure, the first group may be a group of super-agers who have cognitive abilities beyond an average cognitive ability of an age group to which the subjects belong, and the second group may be a group of typical-agers who are not super-agers.

In the present disclosure, the generating of the augmented test data may include generating, by using a preset data generation algorithm, augmented test data including virtual blood test results and cognitive function test results.

In the present disclosure, the predicting of whether or not belonging to the first group may include generating primary prediction results by using at least two different classification algorithms for the augmented test data, and generating a final prediction result by stacking the primary prediction results.

In the present disclosure, the augmented test data may be preprocessed by using LabelEncoder and SimpleImputer before being input to the at least two different classification algorithms.

In the present disclosure, the determined major cognitive function items may be verbal memory, visual memory, and visuospatial items.

In the present disclosure, the blood-based cognitive ability evaluation method may further include generating an evaluation model which evaluates cognitive ability, based on the major blood factors extracted for each of the major cognitive function items.

Provided is a computer-readable recording medium having recorded thereon a program for causing a computer to perform the method of the present disclosure.

According to another embodiment of the present disclosure, a blood-based cognitive ability evaluation device may include: a processor; a communication unit; and a memory, wherein the processor may be configured to obtain actual test data including actual blood test results and cognitive function test results of subjects, determine major cognitive function items of a cognitive function test related to whether the subjects belong to a first group, based on the actual test data, generate augmented test data, based on the obtained actual test data, predict, by using a classification algorithm, whether pieces of individual data of the augmented test data belong to the first group, classify the pieces of individual data of the augmented test data and the actual test data into one of the first group and a second group, and extract one or more major blood factors associated with the first group for each of the major cognitive function items, based on the classified actual test data and the classified augmented test data.

### Advantageous Effects of Invention

According to the problem-solving means of the present disclosure, embodiments of the present disclosure may evaluate cognitive ability of a subject, based on a blood test, and may increase accessibility to cognitive ability evaluation. In addition, the financial burden on the subject may be reduced and the time for cognitive ability tests may be saved.

Furthermore, embodiments of the present disclosure may provide highly reliable evaluation results by evaluating cognitive ability, based on a blood test, and may assist in continuous health management by enabling follow-up observation over time.

Of course, the scope of the present disclosure is not limited by such an effect.

### BRIEF DESCRIPTION OF DRAWINGS

FIG. 1 is a diagram for describing a cognitive ability evaluation system according to an embodiment of the present disclosure.

FIG. 2 is a diagram illustrating an example of test data of FIG. 1.

FIG. 3 is a block diagram schematically illustrating a cognitive ability evaluation device according to an embodiment of the present disclosure.

FIG. 4 is a flowchart for describing a cognitive ability evaluation method according to an embodiment of the present disclosure.

FIG. 5A is a graph showing major blood factors of an attention domain.

FIG. 5B is a graph showing major blood factors of a language domain.

FIG. 5C is a graph showing major blood factors of a verbal memory domain.

FIG. 5D is a graph showing major blood factors of a visual memory domain.

FIG. 5E is a graph showing major blood factors of a visuospatial domain.

FIG. 5F is a graph showing major blood factors of a frontal domain.

FIG. 6 is a flowchart for describing a cognitive ability evaluation method according to another embodiment of the present disclosure.

FIG. 7 is a graph showing a correlation between each item of a cognitive function test and a super-ager group, according to an embodiment of the present disclosure.

FIG. 8 is a graph showing accuracy of a prediction model by a classification algorithm, according to an embodiment of the present disclosure.

FIGS. 9A to 9E are graphs for describing blood factors associated with a first group determined for each item of a cognitive function test.

### MODE FOR THE INVENTION

The advantages and features of the present disclosure, and methods of achieving them will be clarified with reference to embodiments described below in detail with reference to the accompanying drawings. However, the present disclosure is not limited to the embodiments presented below and may be implemented in various different forms. Rather, it will be understood that the present disclosure includes all modifications, equivalents, and substitutes falling within the concept and technical scope of the present disclosure.

The terms as used in the present application are only used to describe particular embodiments and are not intended to limit the present disclosure. The singular forms as used herein are intended to include the plural forms as well unless the context clearly indicates otherwise. The terms “comprise,” “include,” or “have” as used in the present application are inclusive and therefore specify the presence of one or more stated features, integers, steps, operations, elements, components, or any combination thereof, but do not preclude the presence or addition of one or more other features, integers, steps, operations, elements, components, or any combination thereof.

In addition, connecting lines or connecting members illustrated in the drawings are intended to represent functional connections and/or physical or circuit connections. In an actual device, connecting lines or connecting members illustrated in the drawings may represent connections between components by means of a variety of functional, physical, or circuit connections that may be substituted or added.

Because various changes may be made to some embodiments of the present disclosure, specific embodiments will be illustrated in the drawings and described in detail in the detailed description. Effects and features of the present embodiments, and methods of achieving them will be clarified with reference to the detailed description below along with the drawings. However, the present embodiments are not limited to the embodiments disclosed below and may be implemented in various forms.

In the following embodiments, the terms “first,” “second,” etc. are not used in a restrictive sense and are used to distinguish one element from another. In addition, the singular forms as used in the following embodiments are intended to include the plural forms as well unless the context clearly indicates otherwise. Furthermore, it will be understood that the terms “include” and/or “comprise” used in in the following embodiments specify the presence of stated features or elements, but do not preclude the presence or addition of one or more other features or elements. In addition, it will be understood in the following embodiments that when a portion is referred to as being “connected to” another portion, it may be “directly connected to” the other portion or “electrically connected to” the other portion with intervening portions therebetween.

Hereinafter, the following embodiments will be described in detail with reference to the accompanying drawings. When describing the embodiments with reference to the accompanying drawings, the same or corresponding elements are denoted by the same reference numerals and redundant descriptions thereof are omitted.

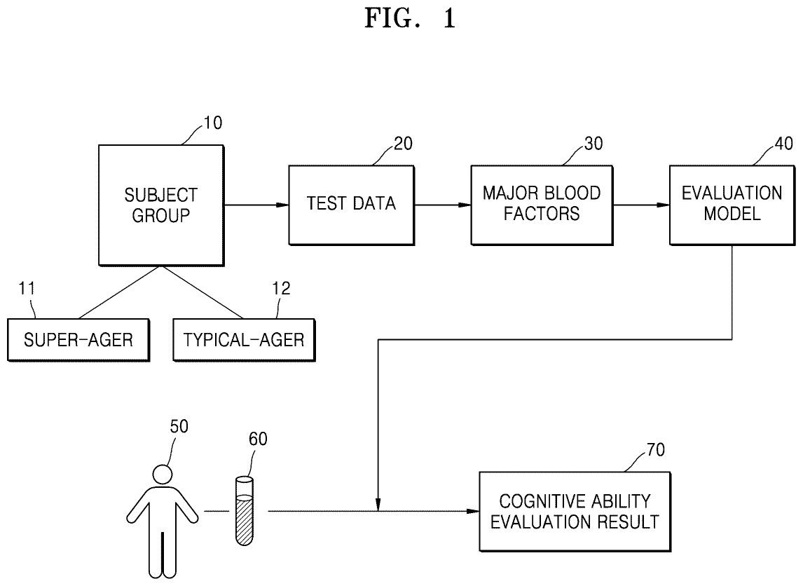

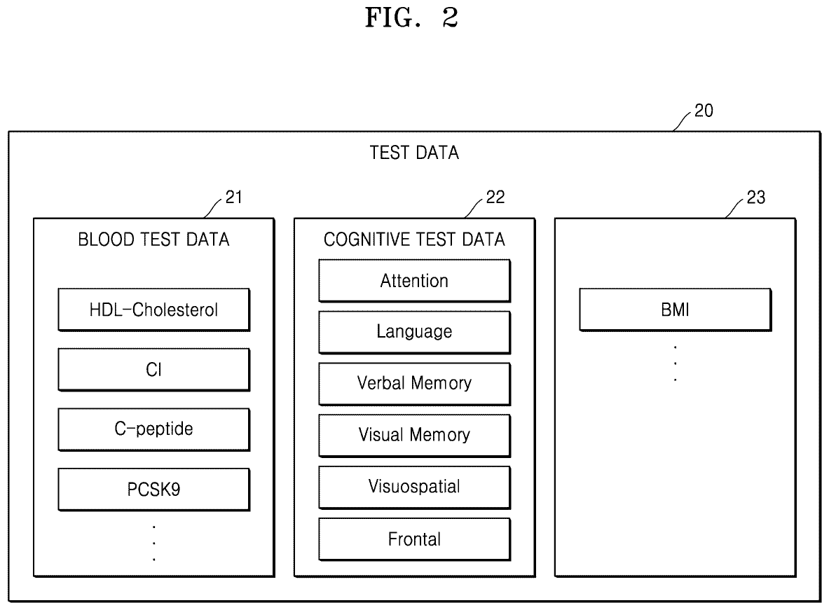

FIG. 1 is a diagram for describing a cognitive ability evaluation system according to an embodiment of the present disclosure, and FIG. 2 is a diagram illustrating an example of test data 20 of FIG. 1.

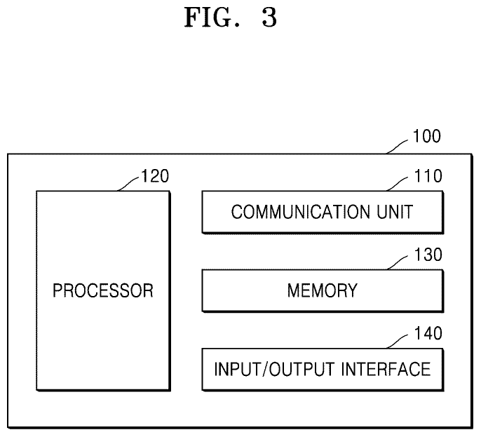

A cognitive ability evaluation server (see 100 of FIG. 3) may obtain the test data 20, including blood test results and cognitive function test results of subjects, from a subject group 10.

Here, the subjects refer to people who are subjected to a blood test and a cognitive function test and provide test results. The subject group including the subjects may be classified into a plurality of age groups, and the respective subjects may be classified into those who have cognitive abilities beyond average cognitive ability of an age group to which the subjects belong and those who do not have cognitive abilities above average cognitive ability of the age group to which the subjects belong.

That is, the subject group including the elder may be classified into a super-ager group and a typical-ager group. In the present specification, the term “super-ager” refers to the elder who have cognitive ability of middle age. The elder are defined as people aged 60 years or older. Of course, the age group which defines the elder may be set to other age ranges. The middle age is defined as the age of 45. Of course, the age group which defines the middle age may be set to other age ranges. The cognitive ability of the subject may be determined through clinical evaluation.

The super-ager is a person with good cognitive reserve and/or brain resilience. The cognitive reserve is a concept based on brain plasticity and means actively coping with changes in the brain that occur with aging. No clear factors that determine cognitive reserve have been identified, but environmental factors such as education levels of subjects are known to affect cognitive reserve. The brain resilience refers to the resilience ability of the brain to repair nerve cells when damaged.

The test data 20 may include blood test data 21, cognitive test data 22, and additional data 23. The blood test data 21 is a blood test result of a subject and may be obtained from a blood test conducted during a general health checkup. For example, the blood test data 21 may include result values of hemoglobin test, fasting blood sugar test, liver function test, serum creatinine test, cholesterol test, and glomerular filtration rate test items, and may include values of factors such as HDL-Cholesterol, CI, C-peptide, and PSK9. However, the present disclosure is not limited thereto, and the blood test data 21 may include result values obtained by various blood test methods.

The cognitive test data 22 is a cognitive function test result of a subject and may include results for a plurality of test items. For example, the cognitive test data 22 may include test results for attention, language, verbal memory, visual memory, visuospatial, and frontal domains.

The test data 20 may include, in addition to the blood test data 21 and the cognitive test data 22, personal data, such as age, gender, or education level, as the additional data 23.

The cognitive ability evaluation device 100 may extract major blood factors 30 from the test data 20 of the subject group 10. Specifically, the cognitive ability evaluation device 100 may extract the major blood factors 30, which influences cognitive ability among a plurality of blood factors included in the blood test data 21, by performing correlation analysis on the blood test data 21 and the cognitive test data 22.

The extracted major blood factors 30 may be used to generate an evaluation model 40 which evaluates cognitive ability of a subject 50. That is, the cognitive ability evaluation device 100 may construct the cognitive ability evaluation model 40 which evaluates cognitive ability by using the major blood factors 30 included in the blood test result.

The cognitive ability evaluation device 100 may extract pieces of blood information including values corresponding to the major blood factors 30 from blood sample data obtained from a blood sample 60 of the subject 50, and may evaluate the cognitive ability of the subject 50 by using the extracted pieces of blood information and the evaluation model 40. The cognitive ability evaluation device 100 may evaluate whether the subject 50 has cognitive ability beyond the average cognitive ability of the age group to which the subject 50 belongs. In addition, the cognitive ability evaluation device 100 may evaluate the cognitive ability of the subject 50 for each item of the cognitive function test.

Hereinafter, an example of the operation of the cognitive ability evaluation device 100 is described with reference to FIGS. 3 to 10.

FIG. 3 is a block diagram schematically illustrating the cognitive ability evaluation device 100 according to an embodiment of the present disclosure.

Referring to FIG. 3, the cognitive ability evaluation device 100 according to an embodiment of the present disclosure may include a communication unit 110, a processor 120, a memory 130, and an input/output interface 140. In the cognitive ability evaluation device 100 illustrated in FIG. 3, only elements related to the present embodiment are illustrated so as to prevent the features of the present embodiment from being ambiguous. Accordingly, it will be understood by those of ordinary skill in the art related to the present embodiment that, in addition to the elements illustrated in FIG. 3, other general-purpose elements may be further included. In addition, it is obvious to those of ordinary skill in the art related to the present embodiment that the communication unit 110, the processor 120, the memory 130, and the input/output interface 140 illustrated in FIG. 3 may be implemented as independent devices.

The cognitive ability evaluation device 100 according to an embodiment of the present disclosure may extract, from test data of a subject, major blood factors used for cognitive ability evaluation, may generate an evaluation model for evaluating cognitive ability, based on the extracted major blood factors, and may evaluate the cognitive ability of the subject by using a blood sample of the subject.

The user may identify the cognitive ability evaluation result of the subject through the cognitive ability evaluation device 100. Here, the user is a person who wants to identify the cognitive ability evaluation result of the subject. For example, the user may be a medical doctor, a clinician, a subject itself, or a manager of a subject.

In an embodiment, the cognitive ability evaluation device 100 may be mounted on a user terminal. In this case, the user terminal may be a mobile terminal or a stationary terminal implemented as a computer device, and may communicate with other user terminals and/or servers through a network by using a wireless or wired communication scheme. For example, the user terminal may be smartphones, mobile phones, navigation systems, computers, laptops, digital broadcasting terminals, personal digital assistants (PDAs), portable multimedia players (PMPs), tablet personal computers (PCs), wearable devices, or the like, but the present disclosure is not limited thereto.

In another embodiment, the cognitive ability evaluation device 100 may be provided in a server. In this case, the server may be implemented as a computer device or a plurality of computer devices which communicate with a plurality of user terminals through a network and provide commands, codes, files, content, services, or the like. For example, the cognitive ability evaluation device 100 may be provided in two servers. The training of the evaluation model which evaluates cognitive ability may be performed through one server, and the cognitive ability of the subject may be evaluated from blood sample data of the subject through the other server, based on a learned algorithm.

In case that the cognitive ability evaluation device 100 is provided in the server, the server may provide a file for installing an application to the user terminal connected through the network. In this case, the user terminal may install the application by using the file provided from the server. In addition, the user terminal may access the server and receive services or content provided by the server under the control of an operating system (OS) and at least one program (e.g., a browser or an installed application), which are included in the user terminal.

The communication unit 110 may provide a configuration or a function which allows the cognitive ability evaluation device 100 to communicate with an external device (not shown) through the network. For example, control signals, commands, data, and the like, which are provided under the control of the processor 120, may be transmitted to the external device through the communication unit 110 and the network.

The communication unit 110 may receive, from the external device, test data including blood test results and cognitive function test results of subjects. In addition, the communication unit 110 may receive blood sample data and additional information of the subject from the external device. Furthermore, the communication unit 110 may transmit cognitive ability evaluation results to the external device such as the user terminal.

The processor 120 may be configured to process commands of a computer program by performing basic arithmetic, logic, and input/output operations. Here, the commands may be provided to the processor 120 by the memory 130 or the communication unit 110. For example, the processor 120 may be configured to execute the received commands according to program code stored in a recording device such as the memory 130. In addition, the processor 120 may control the overall operations of other elements included in the cognitive ability evaluation device 100.

The processor 120 may be implemented as an array of a plurality of logic gates, or may be implemented as a combination of a general-purpose microprocessor and a memory storing a program that is executable on the microprocessor. For example, the processor 120 may include a general-purpose processor, a central processing unit (CPU), a microprocessor, a digital signal processor (DSP), a controller, a microcontroller, a state machine, or the like. In some environments, the processor 120 may include an application-specific integrated circuit (ASIC), a programmable logic device (PLD), a field programmable gate array (FPGA), or the like. For example, the processor 120 may be a combination of processing devices, such as a combination of a DSP and a microprocessor, a combination of a plurality of microprocessors, a combination of one or more microprocessors connected to a DSP core, or a combination of any other configurations.

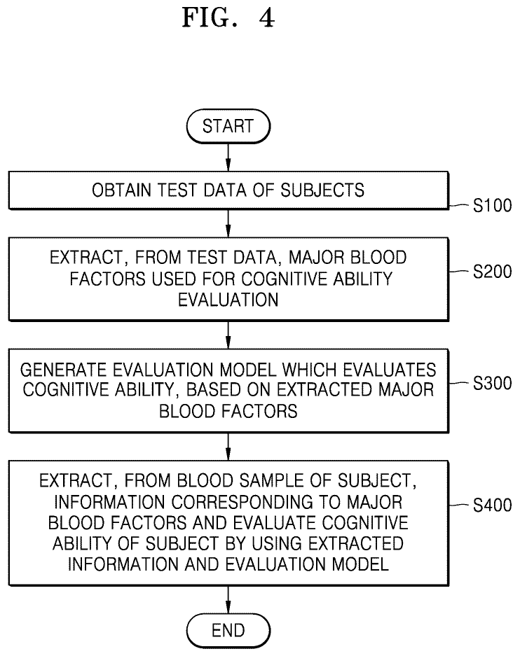

The memory 130 may include any non-transitory computer-readable recording medium. As an example, the memory 130 may include a permanent mass storage device, such as random access memory (RAM), read-only memory (ROM), disk drive, solid state drive (SSD), or flash memory. As another example, the permanent mass storage device, such as ROM, SSD, flash memory, or disk drive, may be a separate permanent storage device, which is distinct from memory. In addition, the memory 130 may store an OS and at least one program code (e.g., code for causing the processor 120 to perform operations to be described below with reference to FIG. 4).

The input/output interface 140 may be a means for interfacing with an input or output device (e.g., a keyboard, a mouse, a touchscreen, etc.) which may be connected to or included in the cognitive ability evaluation device 100. In FIG. 3, the input/output interface 140 is illustrated as an element configured separately from the processor 120, but the present disclosure is not limited thereto, and the input/output interface 140 may be configured to be included in the processor 120.

FIG. 4 is a flowchart for describing a cognitive ability evaluation method according to an embodiment of the present disclosure.

Referring to FIG. 4, the cognitive ability evaluation method according to an embodiment of the present disclosure includes obtaining test data of subjects (S100), extracting, from the test data, major blood factors used for cognitive ability evaluation (S200), generating an evaluation model which evaluates cognitive ability, based on the extracted major blood factors (S300), and extracting, from a blood sample of the subject, information corresponding to the major blood factors and evaluating the cognitive ability of the subject by using the extracted information and the evaluation model (S400). Hereinafter, the technical idea of the present disclosure is described by specifically exemplifying a researcher's actual test.

First, the cognitive ability evaluation method according to an embodiment of the present disclosure includes obtaining the test data of the subjects (S100).

The cognitive ability evaluation device 100 may receive the test data of the subjects from the external device through the communication unit 110 and transmit the received test data to the processor 120.

The test data may include the blood test data 21, the cognitive test data 22, and the additional data 23. The blood test data 21 is the blood test result of the subject and may be obtained from a blood test conducted during a general health checkup. However, the present disclosure is not limited thereto, and the blood test data 21 may include result values obtained by various blood test methods.

The cognitive test data 22 is the cognitive function test result of the subject and may include results for a plurality of test items. For example, the cognitive test data 22 may include test results for attention, language, verbal memory, visual memory, visuospatial, and frontal domains.

The test data 20 may include, in addition to the blood test data 21 and the cognitive test data 22, personal data, such as age, gender, or education level, as the additional data 23, and may also include physical examination data such as body mass index (BMI).

As a specific example, a subject group configured by the researcher consisted of 81 elderly people aged 60 or older, and blood tests and cognitive function tests were conducted on all subjects.

Next, the major blood factors used for cognitive ability evaluation are extracted from the test data (S200).

The processor 120 may perform correlation analysis between the blood test result and the cognitive function test result, which are included in the test data, and may extract the major blood factors to be used for cognitive ability evaluation. In this case, the processor 120 may further use the additional data, which is included in the test data, for the correlation analysis.

Specifically, the processor 120 may classify subjects having cognitive abilities beyond the average cognitive ability of the age group to which the subjects belong, by using the cognitive function test result included in the test data. That is, the subject group is classified into a plurality of age groups, and the respective subjects may be classified into those who have cognitive abilities beyond the average cognitive ability of the age group to which the subjects belong and those who do not have cognitive abilities beyond the average cognitive ability of the age group to which the subjects belong.

In a specific test example, the subject group configured by researcher was classified into a super-ager group of 41 super-agers having cognitive abilities beyond the average cognitive ability of the age group to which the subjects belong and a typical-ager group of 40 typical-agers, by using the cognitive function test result. In the present specification, for convenience of explanation, the super-ager group may be defined as a first group, and the typical-ager group may be defined as a second group.

The results of statistical analysis performed on the cognitive function test results of the super-ager group and the typical-ager group are shown in Table 1 below. The cognitive function test results showed a significant difference between the two groups in language, verbal memory, visual memory, and visuospatial domains, each of which was p<0.05. Here, p represents a significance probability value (p-value). In addition, each of the verbal memory, visual memory, and visuospatial domains was p<0.01, which showed a significant difference between the two groups.

TABLE 1

| | TypicalAgers | SuperAgers | |
|---|---|---|---|
| Variable | (n = 40) | (n = 41) | p* |
| Cognitive Domains | | | |
| Attention | 0.05 ± 0.92 | 0.45 ± 1.04 | 0.074 |
| Language | −0.09 ± 0.90 | 0.33 ± 0.79 | 0.028 |
| VerbalMemory | 0.28 ± 1.03 | 1.05 ± 0.72 | <0.001 |
| VisualMemory | −0.19 ± 0.61 | 0.89 ± 0.91 | <0.001 |
| Visuospatial | −0.19 ± 0.76 | 0.30 ± 0.63 | 0.002 |
| Frontal | 0.58 ± 0.85 | 0.81 ± 0.63 | 0.176 |

The results of statistical analysis performed on the blood test results of the two groups are shown in Table 2 below. The blood test results showed a significant difference between the two groups in High Density Lipoprotein (HDL), CI, C-peptide, and PCSK9, each of which was p<0.05.

TABLE 2

| | Typical Agers | SuperAgers | | | Typical Agers | SuperAgers | |
|---|---|---|---|---|---|---|---|
| Variable | (n = 40) | (n = 41) | p* | Variable | (n = 40) | (n = 41) | p* |
| Blood Analysis | | | | Blood Analysis | | | |
| HbA1c | 5.90 ± 0.72 | 5.80 ± 0.63 | 0.285 | C-peptide | 2.29 ± 1.15 | 1.86 ± 0.63 | 0.039 |
| Total calcium | 9.29 ± 0.31 | 9.35 ± 0.38 | 0.469 | Folic acid | 15.06 ± 9.24 | 14.92 ± 7.40 | 0.942 |
| Phosphorus | 3.70 ± 0.43 | 3.64 ± 0.37 | 0.552 | Vit B12 | 868.80 ± 552.97 | 862.37 ± 300.70 | 0.948 |
| Glucose | 101.90 ± 18.36 | 102.85 ± 20.69 | 0.827 | WBC | 5.50 ± 1.20 | 5.14 ± 1.16 | 0.17 |
| BUN | 16.48 ± 4.27 | 16.20 ± 4.46 | 0.774 | RBC | 4.36 ± 0.49 | 4.28 ± 0.44 | 0.449 |
| Creatinine | 0.77 ± 0.19 | 0.74 ± 0.18 | 0.481 | Hb | 13.38 ± 1.39 | 13.08 ± 1.09 | 0.281 |
| eGFR-MDRD (IDMS) | 83.05 ± 18.75 | 87.24 ± 19.51 | 0.327 | Hct | 39.36 ± 3.91 | 38.50 ± 3.33 | 0.29 |
| eGFR-CKD-EPl | 80.68 ± 13.93 | 83.12 ± 11.23 | 0.386 | MCV | 90.49 ± 2.88 | 90.17 ± 3.36 | 0.643 |
| Triglyceride | 110.95 ± 46.13 | 102.44 ± 44.31 | 0.4 | MCH | 30.75 ± 1.12 | 30.65 ± 1.29 | 0.71 |
| Total Cholesterol | 164.38 ± 36.12 | 177.63 ± 42.98 | 0.137 | MCHC | 33.99 ± 0.90 | 33.99 ± 0.79 | 0.985 |
| Total Protein | 7.07 ± 0.32 | 7.08 ± 0.41 | 0.852 | RDW(CV) | 12.65 ± 0.49 | 12.69 ± 0.63 | 0.725 |
| Albumin | 4.48 ± 0.21 | 4.54 ± 0.22 | 0.152 | Platelet | 217.59 ± 50.08 | 212.60 ± 42.78 | 0.631 |
| AST | 25.95 ± 15.58 | 24.39 ± 6.88 | 0.56 | ELISA result | | | |
| ALT | 21.55 ± 11.6 | 19.29 ± 7.79 | 0.306 | PCSK9 (ng/ml) | 99.30 ± 35.53 | 116.60 ± 42.28 | 0.05 |
| ALP | 70.23 ± 21.82 | 70.98 ± 20.64 | 0.874 | ApoE (ug/ml) | 114.34 ± 75.17 | 96.76 ± 46.41 | 0.208 |
| Total Bilirubin | 0.61 ± 0.22 | 0.64 ± 0.26 | 0.67 | CD36 (ng/ml) | 105.43 ± 103.10 | 152.22 ± 169.83 | 0.138 |
| HDL-Cholesterol | 54.33 ± 12.02 | 60.15 ± 11.17 | 0.027 | EPO (mIU/ml) | 10.48 ± 3.74 | 12.19 ± 4.84 | 0.079 |
| LDL-Cholesterol | 95.33 ± 32.80 | 102.83 ± 37.52 | 0.341 | AGE (ng/ml) | 12.92 ± 5.45 | 12.92 ± 7.58 | 0.999 |
| Na | 141.45 ± 2.19 | 141.24 ± 1.68 | 0.637 | RAGE (pg/ml) | 82.31 ± 26.39 | 84.31 ± 28.54 | 0.744 |
| K | 4.48 ± 0.46 | 4.40 ± 0.35 | 0.351 | Vimentin (ng/ml) | 172.10 ± 163.97 | 163.46 ± 88.99 | 0.768 |
| Cl | 105.90 ± 2.47 | 104.83 ± 2.14 | 0.04 | FABP-3 (pg/ml) | 1218.80 ± 811.65 | 1215.94 ± 548.00 | 0.985 |
| CRP | 0.11 ± 0.15 | 0.13 ± 0.13 | 0.68 | FABP-4 (ng/ml) | 29.33 ± 13.49 | 29.84 ± 14.13 | 0.868 |
| TSH | 2.16 ± 1.28 | 2.45 ± 2.00 | 0.442 | oxLDL (ng/ml) | 51.77 ± 5.17 | 51.66 ± 3.68 | 0.913 |
| Free T4 | 1.28 ± 0.39 | 1.20 ± 0.17 | 0.224 | Adioponectin (ug/ml) | 35.98 ± 25.16 | 43.71 ± 41.15 | 0.31 |
| Insulin | 9.21 ± 6.51 | 7.63 ± 4.92 | 0.219 | Leptin (ng/ml) | 7.80 ± 4.86 | 8.41 ± 6.60 | 0.683 |

In addition, the correlation analysis was performed between the cognitive function test result and the blood test result of the subject group, and the features significantly derived for each item of the cognitive function test are as follows.

The attention domain showed a significant correlation with HDL cholesterol (r=0.254, p=0.022) and insulin (r=−0.223, p=0.046). The language domain showed a significant correlation with mean corpuscular hemoglobin concentration (MCHC) (r=0.261, p=0.019). The verbal memory domain showed a significant correlation with C-peptide (r=−0.281, p=0.011), erythropoietin (EPO) (r=0.227, p=0.042), and HDL cholesterol (r=0.219, p=0.049). The visual memory domain showed a significant correlation with PCSK9 (r=0.281, p=0.011), HDL cholesterol (r=0.264, p=0.017), and free T4 (r=−0.234, p=0.036). The visuospatial domain showed a significant correlation with HDL cholesterol (r=0.341, p=0.002), triglyceride (r=−0.332, p=0.002), free T4 (r=−0.326, p=0.003), apolipoprotein E (ApoE) (r=−0.287, p=0.009), insuline (r=−0.231, p=0.038), and aspartate aminotransferase (AST) (r=−0.228, p=0.041). The frontal domain showed a significant correlation with oxidized low density lipoprotein (oxLDL) (r=−0.356, p=0.001), hemoglobin (Hb) (r=−0.288, p=0.009), hematocrit (Hct) (r=−0.262, p=0.018), vimentin (r=−0.246, p=0.027), K (r=0.237, p=0.034), red blood cell (RBC) (r=−0.227, p=0.041), and creatinine (r=−0.226, p=0.042). Here, r represents a Pearson correlation coefficient.

The processor 120 may perform correlation analysis between the respective variables included in the test data by using the test data of the super-ager group and the typical-ager group classified from the subject group, and may extract major blood factors to be used for cognitive ability evaluation, based on a preset variable selection algorithm (i.e., perform variable selection (feature selection)). In this case, the number of extracted major blood factors may be 10, but the present disclosure is not limited thereto.

In case that an evaluation model is generated by using machine learning, variables (features or attributes) are used. However, in case that a large number of variables or inappropriate variables are used, a machine learning model may overfit, or the prediction accuracy may decrease. Therefore, in order for the machine learning model to have high prediction accuracy, it is necessary to use an appropriate combination of variables. That is, by selecting variables most highly associated with prediction variables to be predicted, the complexity of the machine learning model may be reduced while using as few variables as possible.

The variable selection algorithm may be, for example, a selectKBest algorithm, but the present disclosure is not limited thereto.

The major blood factors, which are extracted from the test data by using the preset variable selection algorithm, may include at least one of hemoglobin A1c (HbA1c), total calcium, phosphorus, glucose, blood urea nitrogen BUN), creatinine, eGFRMDRD, eGFRCKDEPI, triglyceride, total cholesterol, total protein, albumin, AST, alanine aminotransferase (ALT), alkaline phosphatase (ALP), total bilirubin, HDLCholesterol, LDLCholesterol, sodium (Na), potassium (K), chlorine (CI), C-reactive protein (CRP), thyroid-stimulating hormone (TSH), free T4, insulin, C-peptide, folic acid, Vit B12, white blood cell (WBC), RBC, Hb, Hct, mean corpuscular volume (MCV), mean corpuscular hemoglobin (MCH), MCHC, red cell distribution width (coefficient of variation) (RDW(CV)), platelet, PCSK9, ApoE, CD36, EPO, advanced glycation end products (AGE), receptor for advanced glycation end products (RAGE), vimentin, FABP-3, FABP-4, oxLDL, adioponectin, or leptin.

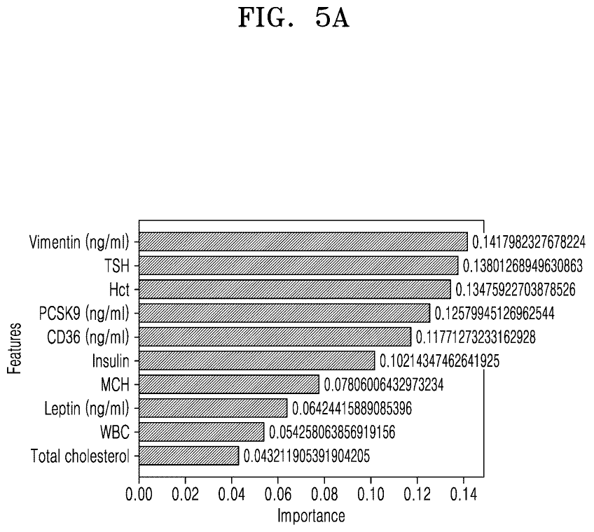

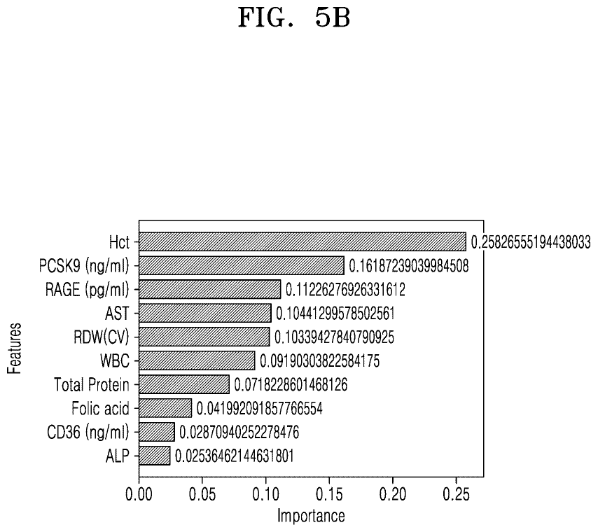

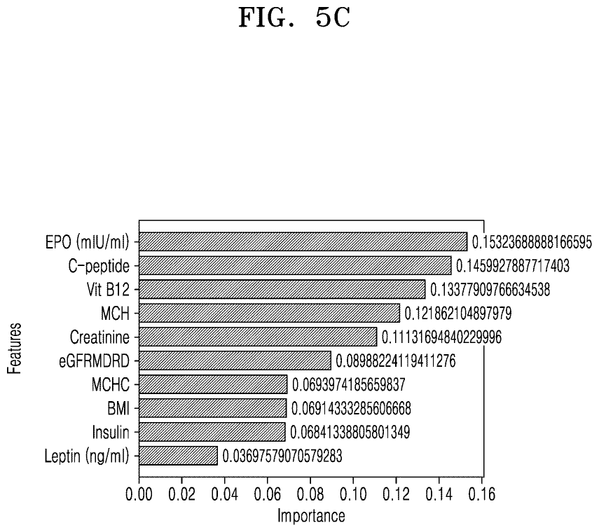

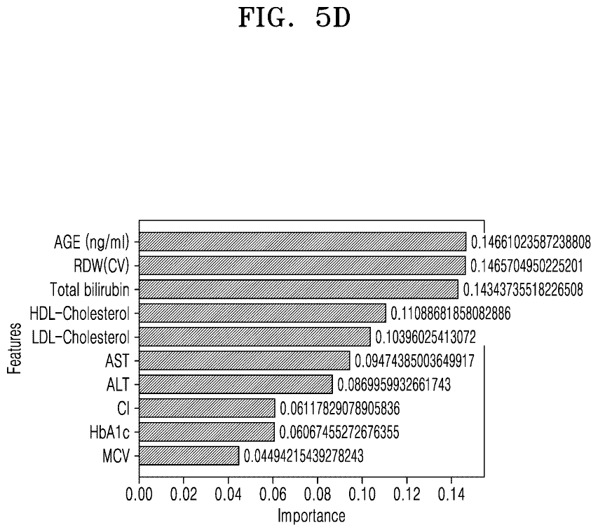

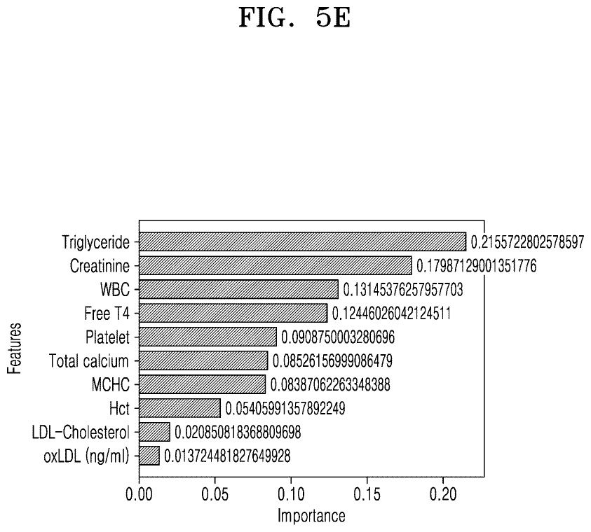

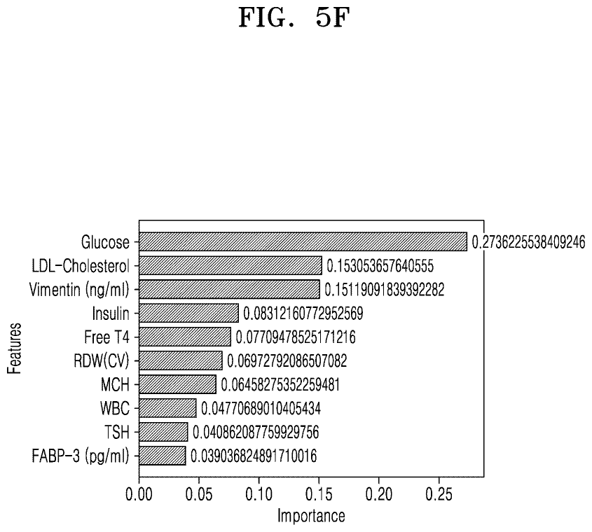

On the other hand, the processor 120 may extract the major blood factors for each item of the cognitive function test by using the preset variable selection algorithm (according to an embodiment, selectKBest). Hereinafter, examples of the major blood factors selected for each item of the cognitive function test are described with reference to FIGS. 5A to 5E.

FIG. 5A is a graph showing major blood factors of an attention domain, FIG. 5B is a graph showing major blood factors of a language domain, FIG. 5C is a graph showing major blood factors of a verbal memory domain, FIG. 5D is a graph showing major blood factors of a visual memory domain, FIG. 5E is a graph showing major blood factors of a visuospatial domain, and FIG. 5F is a graph showing major blood factors of a frontal domain. In each of the graphs, importance of each variable is calculated based on the degree of contribution to evaluating cognitive ability in each item of the cognitive function test, and the top 10 variables with the highest importance are graphically displayed.

Referring to FIG. 5A, the major blood factors of the attention domain are extracted as total calcium, triglyceride, total cholesterol, total bilirubin, HDL-cholesterol, Na, TSH, CD36, EPO, and leptin.

Referring to FIG. 5B, the major blood factors of the language domain are extracted as glucose, insulin, C-peptide, RBC, Hb, MCH, MCHC, RAGE, adiponectin, and leptin.

Referring to FIG. 5C, the major blood factors of the verbal memory domain are extracted as HbA1c, Glucose, ALT, HDL-cholesterol, Free-T4, C-peptide, MCV, MCH, Platelet, and EPO.

Referring to FIG. 5D, the major blood factors of the visual memory domain are extracted as HbA1c, total cholesterol, HDL-cholesterol, LDL-cholesterol, free T4, WBC, RBC, Hct, PCSK9, and oxLDL.

Referring to FIG. 5E, the major blood factors of the visuospatial domain are extracted as HbA1c, BUN, triglyceride, total cholesterol, AST, HDL-cholesterol, free T4, insulin, C-peptide, and ApoE.

Referring to FIG. 5F, the major blood factors of the frontal domain are extracted as triglyceride, total cholesterol, K, Hb, Hct, MCH, MCHC, CD36, AGE, and oxLDL.

FIGS. 5A to 5E illustrate examples of the major blood factors extracted for each item of the cognitive function test, and the present disclosure is not limited thereto.

Next, the evaluation model which evaluates cognitive ability is generated based on the extracted major blood factors (S300).

The processor 120 may train the evaluation model by using the major blood factors and may generate the evaluation model which evaluates cognitive ability. The evaluation model may be trained to receive blood sample data from the subject and output the cognitive ability evaluation result of the subject.

On the other hand, in the generating of the evaluation model (S300), the processor 120 may generate augmented data, based on the test data of the actual subjects, and train the evaluation model, so as to solve the quantitative problem of the test data (training data) obtained from the subject group. In the present specification, for the purpose of distinguishing data, the test data obtained from the actual subjects may be referred to as actual test data, and the generated augmented data may be referred to as augmented test data.

According to an embodiment, the processor 120 may generate augmented test data by using a preset augmented data generation algorithm. As the augmented data generation algorithm, Generation of REAlistic Tabular data (GReaT), which is a large language model-based tabular data augmentation, may be used, but the present disclosure is not limited thereto. As a specific example, the researcher generated 729 pieces of augmented data by using a large language model (LLM)-based algorithm, and an evaluation model for each cognitive function item was trained by using a total of 810 pieces of data.

The evaluation model according to an embodiment of the present disclosure may be a statistical learning algorithm which is implemented based on a biological neural network structure in machine learning technology and cognitive science, or a machine learning model which executes the algorithm. For example, the machine learning model may represent a model with problem-solving capabilities, which is trained such that nodes, which are artificial neurons that form a network through a combination of synapses as in a biological neural network, repeatedly adjust weights of the synapses so as to reduce an error between a correct output corresponding to a specific input and an inferred output. For example, the machine learning model may be implemented as multilayer perceptron (MLP) including multilayer nodes and connections therebetween.

The evaluation model according to the present embodiment may be implemented by using one of various artificial neural network model structures including MLP. For example, the evaluation model may include an input layer which receives input signals or data from the outside, an output layer which outputs output signals or data corresponding to the input data, and at least one hidden layer which is located between the input layer and the output layer and receives signals from the input layer, extracts features, and transmits the extracted features to the output layer. The output layer receives signals or data from the hidden layer and outputs the received signals or data to the outside.

For example, the evaluation model may be generated by using at least one of linear regression, Lasso, decision tree, random forest (RF), support vector machine (SVM), GradientBoost, AdaBoost, or XgBoost, but the present disclosure is not limited thereto.

On the other hand, as described above, in the generating of the evaluation model (S300), the evaluation model may be verified by using the augmented data generated based on the test data. More specifically, the processor 120 may generate the augmented data based on the test data obtained from the subject group. As an example, an LLM-based GReaT model may be used as an augmented data generation algorithm.

As a specific example, the researcher generated 729 pieces of augmented data by using a GReaT algorithm and was able to perform 10-fold cross-validation for each learning model by using a total of 810 pieces of data. As a result of the cross-validation, training was performed with the 729 pieces of augmented data and validation was performed with 81 pieces of existing data. Hyperparameter tuning was performed by using Pycaret, which is well known as automatic machine learning (Auto ML). Mean square error (MSE), root mean square error (RMSE), and mean absolute error (MAE) were used as evaluation functions.

[Table 3] below shows error rates for 17 regression analysis algorithms (Linear, Least Angle, Lasso, Lasso Least Angle, Ridge, Bayesian Ridge, Orthogonal Matching Pursuit, Decision Tree, Extra Trees, Random Forest, Huber, Elastic Net, Passive Aggressive, K Neighbors, AdaBoost, Gradient Boost, and Light Gradient Boost) for each cognitive domain. Referring to [Table 3], it may be seen that the augmented data shows an average error rate of 0.15 based on MAE.

TABLE 3

| | Attention | | | Language | | | Verbal Memory | | |
|---|---|---|---|---|---|---|---|---|---|
| Regression Model | MSE | R.MSE | MAE | MSE | RMSE | MAE | MSE | RMSE | MAE |
| Linear | 0.0422 | 0.2053 | 0.1625 | 0.0265 | 0.1629 | 0.1397 | 0.0500 | 0.2237 | 0.1806 |
| Least Angle | 0.0422 | 0.2053 | 0.1625 | 0.0265 | 0.1629 | 0.1397 | 0.0500 | 0.2237 | 0.1806 |
| Lasso | 0.0437 | 0.2090 | 0.1667 | 0.0266 | 0.1630 | 0.1400 | 0.0479 | 0.2189 | 0.1770 |
| Lasso Least Angle | 0.0437 | 0.2090 | 0.1667 | 0.0266 | 0.1630 | 0.1400 | 0.0555 | 0.2357 | 0.1892 |
| Ridge | 0.0424 | 0.2059 | 0.1635 | 0.0266 | 0.1632 | 0.1398 | 0.0499 | 0.2234 | 0.1805 |
| Bayesian Ridge | 0.0429 | 0.2071 | 0.1650 | 0.0266 | 0.1631 | 0.1400 | 0.0485 | 0.2203 | 0.1782 |
| Orthogonal Matching Pursuit | 0.0436 | 0.2088 | 0.1663 | 0.0265 | 0.1626 | 0.1389 | 0.0488 | 0.2209 | 0.1783 |
| Decision Tree | 0.0450 | 0.2122 | 0.1678 | 0.0276 | 0.1661 | 0.1424 | 0.1017 | 0.3190 | 0.2560 |
| Extra Trees | 0.0450 | 0.2122 | 0.1678 | 0.0291 | 0.1704 | 0.1423 | 0.0502 | 0.2240 | 0.1768 |
| Random Forest | 0.0437 | 0.2091 | 0.1654 | 0.0287 | 0.1694 | 0.1467 | 0.0512 | 0.2263 | 0.1788 |
| Huber | 0.0428 | 0.2068 | 0.1621 | 0.0264 | 0.1624 | 0.1395 | 0.0496 | 0.2227 | 0.1799 |
| Elastic Net | 0.0437 | 0.2090 | 0.1667 | 0.0266 | 0.1630 | 0.1400 | 0.0479 | 0.2189 | 0.1770 |
| Passive Aggressive | 0.0565 | 0.2378 | 0.1902 | 0.0344 | 0.1856 | 0.1589 | 0.0616 | 0.2482 | 0.2049 |
| K Neighbors | 0.0433 | 0.2082 | 0.1678 | 0.0274 | 0.1655 | 0.1416 | 0.0541 | 0.2326 | 0.1867 |
| AdaBoost | 0.0431 | 0.2076 | 0.1676 | 0.0272 | 0.1650 | 0.1422 | 0.0498 | 0.2232 | 0.1789 |
| Gradient Boosting | 0.0437 | 0.2090 | 0.1679 | 0.0294 | 0.1716 | 0.1493 | 0.0534 | 0.2311 | 0.1870 |
| Light Gradient Boosting | 0.0437 | 0.2090 | 0.1667 | 0.0266 | 0.1630 | 0.1400 | 0.0585 | 0.2419 | 0.1913 |
| | Visual Memory | | | Visuospatial | | | Frontal | | |
| Regression Model | MSE | RMSE | MAE | MSE | RMSE | MAE | MSE | RMSE | MAE |
| Linear | 0.0405 | 0.2013 | 0.1605 | 0.0361 | 0.1901 | 0.1472 | 0.0247 | 0.1570 | 0.1241 |
| Least Angle | 0.0397 | 0.1991 | 0.1606 | 0.0350 | 0.1871 | 0.1519 | 0.0247 | 0.1570 | 0.1241 |
| Lasso | 0.0390 | 0.1975 | 0.1569 | 0.0361 | 0.1900 | 0.1542 | 0.0246 | 0.1568 | 0.1231 |
| Lasso Least Angle | 0.0390 | 0.1975 | 0.1569 | 0.0343 | 0.1852 | 0.1483 | 0.0246 | 0.1568 | 0.1231 |
| Ridge | 0.0390 | 0.1975 | 0.1569 | 0.0343 | 0.1852 | 0.1483 | 0.0245 | 0.1565 | 0.1232 |
| Bayesian Ridge | 0.0395 | 0.1987 | 0.1596 | 0.0335 | 0.1831 | 0.1478 | 0.0244 | 0.1562 | 0.1226 |
| Orthogonal Matching Pursuit | 0.0398 | 0.1996 | 0.1615 | 0.0350 | 0.1871 | 0.1519 | 0.0245 | 0.1565 | 0.1222 |
| Decision Tree | 0.0403 | 0.2007 | 0.1635 | 0.0363 | 0.1905 | 0.1547 | 0.0245 | 0.1565 | 0.1230 |
| Extra Trees | 0.0403 | 0.2007 | 0.1635 | 0.0363 | 0.1905 | 0.1547 | 0.0245 | 0.1565 | 0.1230 |
| Random Forest | 0.0398 | 0.1995 | 0.1565 | 0.0348 | 0.1865 | 0.1403 | 0.0245 | 0.1565 | 0.1230 |
| Huber | 0.0398 | 0.1995 | 0.1565 | 0.0348 | 0.1866 | 0.1403 | 0.0243 | 0.1558 | 0.1235 |
| Elastic Net | 0.0390 | 0.1976 | 0.1594 | 0.0346 | 0.1860 | 0.1474 | 0.0246 | 0.1568 | 0.1231 |
| Passive Aggressive | 0.0390 | 0.1974 | 0.1569 | 0.0345 | 0.1857 | 0.1487 | 0.0259 | 0.1609 | 0.1283 |
| K Neighbors | 0.0408 | 0.2020 | 0.1615 | 0.0351 | 0.1874 | 0.1506 | 0.0254 | 0.1594 | 0.1260 |
| AdaBoost | 0.0390 | 0.1975 | 0.1569 | 0.0343 | 0.1851 | 0.1477 | 0.0242 | 0.1556 | 0.1219 |
| Gradient Boosting | 0.0398 | 0.1995 | 0.1565 | 0.0348 | 0.1865 | 0.1403 | 0.0244 | 0.1563 | 0.1225 |
| Light Gradient Boosting | 0.0398 | 0.1996 | 0.1641 | 0.0497 | 0.2230 | 0.1923 | 0.0246 | 0.1568 | 0.1231 |

In addition, the processor 120 may measure the influence of blood factors on predicting scores for each item of cognitive ability so as to construct an evaluation model. More specifically, the influence of 10 blood factors derived previously on each item of cognitive ability may be evaluated by using SHapley Additive explanation (SHAP). In a specific embodiment, the processor 120 may derive a blood factor which has a greater influence among the 10 factors with respect to each item of cognitive ability. In addition, the processor 120 may derive whether the level of each blood factor influences the increase in cognitive ability score depending on whether the level of each blood factor is high or low. For example, the processor 120 may determine that leptin among the 10 blood factors has the greatest influence in the attention item by using SHAP, and that as the leptin level is lower, the attention score is higher.

Next, blood information corresponding to major blood factors is extracted from blood sample data obtained from the subject, and the cognitive ability of the subject is evaluated by using the extracted blood information and the evaluation model (S400).

The processor 120 may evaluate the cognitive ability of the subject by using the evaluation model. In addition, the processor 120 may evaluate the cognitive ability of the subject for each item of the cognitive function test by using the evaluation model. Furthermore, the processor 120 may use the evaluation model to evaluate whether the subject has cognitive ability beyond the average cognitive ability of the age group to which the subject belongs.

Specifically, the processor 120 may obtain the blood sample data generated from the blood test result of the blood sample of the subject. The processor 120 may extract values corresponding to the major blood factors from the blood sample data and may extract blood information including the major blood factor values.

The processor 120 may input the extracted blood information to the evaluation model and may output the cognitive ability evaluation result of the subject. The cognitive ability evaluation result, which is output from the processor 120, may be scores for each item of the cognitive function test, but the present disclosure is not limited thereto.

In another embodiment, the processor 120 may evaluate the cognitive ability of the subject by calculating a cognitive function score of the subject, comparing the calculated cognitive function score with the average cognitive function score of the age of the subject, and determining whether the subject has cognitive ability beyond the average of the age of the subject. In this case, the processor 120 may output, as a probability value, a degree to which the cognitive ability of the subject deviates from the average cognitive ability of the age of the subject.

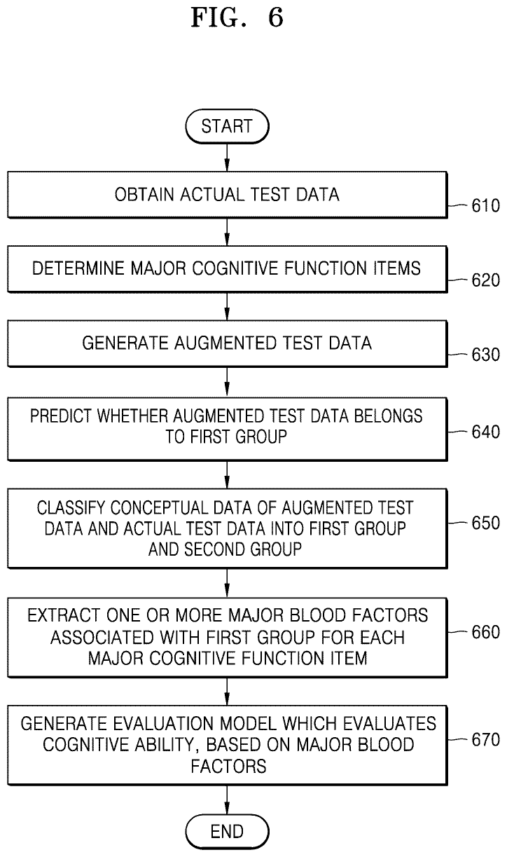

FIG. 6 is a flowchart for describing a cognitive ability evaluation method according to another embodiment of the present disclosure.

First, the cognitive ability evaluation method of the present disclosure includes obtaining actual test data including blood test results and cognitive function test results of the subjects (610). Operation 610 may be similar to operation S100 of FIG. 4 described above, and a redundant description thereof is omitted. As in the test example described in operation S100 of FIG. 4, the subject group includes 81 elderly people aged 60 or older, and the actual test data may be blood test data and cognitive function test data of the tests conducted on the actual subject group.

In relation to the embodiment of FIG. 4 described above, operations 620 to 660 to be described below may replace operations S200 and S300 of FIG. 4. Details thereof are described below.

Next, the cognitive ability evaluation method of the present disclosure includes determining major cognitive function items of the cognitive function test related to whether the subjects belong to a first group, based on the actual test data (620). That is, according to an embodiment, a correlation relationship between each item of the cognitive function test and whether the subject is the super-ager may be trained based on the actual test results. In this regard, according to an embodiment of the present disclosure, all subjects may be classified into a super-ager group (group 1) and a typical-ager group (group 2). Therefore, a correlation between the scores for each item of the cognitive function test of the individual subjects and whether the subject belongs to the super-ager group may be derived based on the actual test results of the subjects classified into the two groups. To this end, according to an embodiment of the present disclosure, a correlation between each item of the cognitive function test and whether the subjects belong to the first group may be trained based on the actual test data and the data classifying the subjects into the first or second group, and the major cognitive function items may be determined based on the training result.

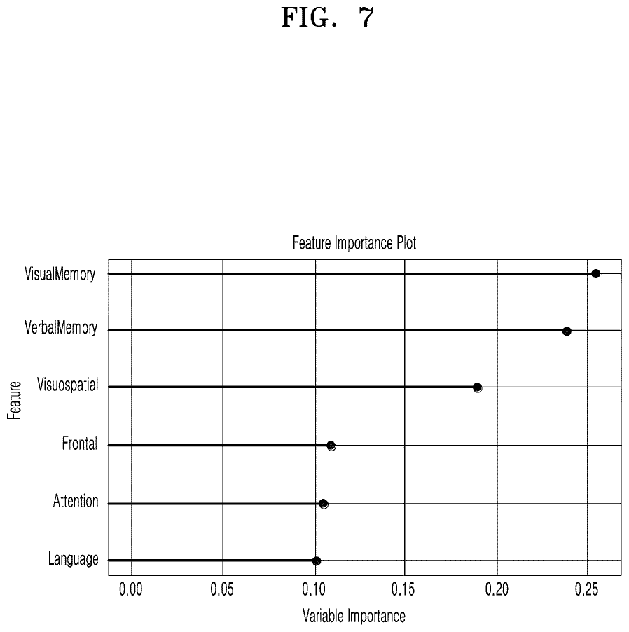

FIG. 7 is a graph showing a correlation between each item of a cognitive function test and a super-ager group, according to an embodiment of the present disclosure.

Referring to FIG. 7, the horizontal axis of the graph represents importance (variable Importance) indicating a degree to which each item is associated with whether the subject is the super-ager, and the vertical axis of the graph represents each item of the cognitive function test as an evaluation factor (feature). As shown in FIG. 7, items with a high degree of association with whether the subject is the super-ager, i.e., major cognitive function items, may be verbal memory, visual memory, and visuospatial items.

In this regard, as described above in operation S200 of FIG. 4, the processor 120 may perform correlation analysis between the blood test result and the cognitive function test result, which are included in the test data, and may extract major blood factors to be used for cognitive ability evaluation. As shown in [Table 1], each of verbal memory, visual memory, and visuospatial domains in the cognitive function test was p<0.01, which showed a significant difference between the two groups. It may be seen that this result is consistent with the importance of each item of the cognitive function test according to the embodiment of FIG. 7.

Next, the cognitive function evaluation method of the present disclosure includes generating augmented test data which may supplement the actual test data (630). More specifically, the cognitive function evaluation method of the present disclosure may include generating augmented test data, including a virtual blood test result and a cognitive function test result, by using a preset data generation algorithm. In this regard, as described above, the generating of the evaluation model (S300) may generate the augmented data based on the actual test data by using the data generation algorithm and may generate (train) or verify the evaluation model. The augmented data generated from the actual test data may be referred to as augmented test data. According to an embodiment, 729 pieces of augmented test data may be generated from 81 pieces of actual test data.

Next, the cognitive function evaluation method of the present disclosure includes predicting whether pieces of individual data of the augmented test data belong to the first group by using a classification algorithm (640). That is, the result of whether the generated augmented test data belongs to the super-ager group may be predicted. In operations 610 and 620 described above, the result of classifying actual individual subjects into the super-ager group and the typical-ager group may be used. However, because the augmented test data are not for actual subjects, there is no information about whether the individual augmented test data belong to the super-ager group. Therefore, to classify the augmented test data into the super-ager group and the typical-ager group, that is, to predict whether individual augmented test data correspond to the super-ager, labeling whether the augmented test data corresponds to the super-ager is required after a separate classification task is performed.

To this end, according to an embodiment of the present disclosure, primary prediction results may be generated by using different classification algorithms to predict whether respective augmented test data belong to the super-ager group, and then, a final prediction result may be generated by stacking the primary prediction results. According to a specific embodiment, the primary prediction results may be generated by using the classification algorithms of ExtraTreesClassifier, RandomForestClassifier, and GradientBoosting Classifier, but the present disclosure is not necessarily limited thereto, and other classification algorithms may be used to generate the primary prediction results. More specifically, ExtraTreesClassifier is a model which uses Extremely Randomized Trees, which allows predictions to be made by constructing trees for some randomly selected elements. In addition, RandomForestClassifier is a model in which a plurality of decision trees are ensembled, which allows respective trees to perform independent training and prediction and aggregate and predict results thereof. In addition, GradientBoosting Classifier may improve prediction performance by generating a new tree which compensates for an error of a previous tree.

Furthermore, according to an embodiment of the present disclosure, after primary prediction results are generated by using two or more different classification algorithms, a meta model may be trained by using the prediction results as new elements, and the meta model may generate a final prediction result by using the prediction results as input. Accordingly, according to an embodiment of the present disclosure, a prediction model may be provided which provides a prediction result on whether augmented test data belongs to a super-ager group, with improved overall prediction performance by complementing the weaknesses of each classifier.

On the other hand, according to an embodiment of the present disclosure, after preprocessing is performed on the augmented test data, the preprocessed augmented test data may be input to the classification algorithms of the prediction model described above. More specifically, the preprocessing may be performed on the augmented test data to convert categorical data into numerical data through LabelEncoder and to replace missing values with other values, such as mean, median, and mode, through SimpleImputer. That is, LabelEncoder handles encoding of categorical data and SimpleImputer handles missing values appropriately to maintain the completeness of data, and thus, the accuracy of the prediction model by the classification algorithm according to an embodiment of the present disclosure may be increased.

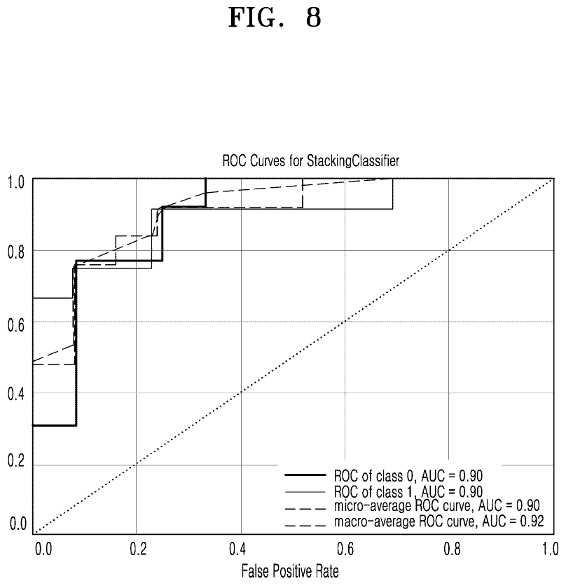

FIG. 8 is a graph showing the accuracy of the prediction model by the classification algorithm, according to an embodiment of the present disclosure.

The graph of FIG. 8 is a receiver operating characteristic (ROC) curve for the prediction model of the present disclosure, and the ROC curve is a graph used to visualize the performance of a binary classification model. In the ROC graph, the x-axis (false positive rate (FPR)) represents a rate of cases where the prediction model of the present disclosure predicted the result as positive when the result was actually negative. Mathematically, FPR=FP/(FP+TN). Here, FP is false positive (the number of cases where the prediction model predicted the result as positive but the result was actually negative), and TN is true negative (the number of cases where the prediction model predicted the result as negative and the result was actually negative). In addition, the Y-axis (true positive rate (TPR)) represents a rate of cases where the result is actually positive and the model correctly predicted the result as positive. Mathematically, TPR=TP/(TP+FN). Here, TP is true positive (the number of cases where the model correctly predicted the result as positive), and FN is false negative (the number of cases where the model predicted the result as negative but the result was actually positive).

Referring to FIG. 8, the ROC curve according to the classification model of the present disclosure may evaluate the performance of the model at various thresholds. Each curve shown in the graph of FIG. 8 may represent a combination of the FPR and the TPR obtained by the prediction model at a specific threshold. More specifically, as the curve is closer to the upper left, the prediction model may be determined to have better performance. This means that the prediction model has a low FPR while maintaining a high TPR. In this regard, as an area under the curve (AUC) is larger, the model may be evaluated as having excellent performance. In the embodiment of FIG. 8, the AUC values of the ROC curves are all 0.9, and thus, it may be evaluated that the prediction model of the present disclosure has high accuracy.

In addition, in an additional embodiment of FIG. 8, a micro-average ROC curve may be used to evaluate the accuracy of the prediction model. The micro-average ROC curve refers to calculating one ROC curve for all classes, instead of drawing an ROC curve for each class in multi-class classification. That is, the micro-average ROC curve may calculate an overall average ROC curve by using the sum of the FP, TP, and FN of all classes. In addition, a macro-average ROC curve is a curve obtained by calculating the ROC curves individually for each class and then averaging the ROC curves. In the embodiment of FIG. 8, because the AUC values of the micro-average ROC curve and the macro-average ROC curve are both 0.9 or more, it may be evaluated that the prediction model of the present disclosure has high accuracy. That is, according to an embodiment of the present disclosure, whether pieces of individual data of the augmented test data belong to the super-ager group may be predicted with an accuracy of about 90%.

Next, the cognitive ability evaluation method of the present disclosure includes classifying actual test data and the augmented test data into a first group and a second group (650). In the following specification, for convenience of explanation, the actual test data and the augmented test data may be collectively referred to as entire test data. As described above, in the determining of the major items of the cognitive function test (620), the respective subjects are divided into the first group or the second group, and thus, the actual test data may be classified into the first group or the second group. In addition, in the predicting of whether the augmented test data belongs to the first group (640), the classification algorithm is used to predict whether the respective augmented test data belong to the first group, and thus, the augmented test data may also be classified into the first group or the second group, based on the prediction result. In a specific example, in case that there are 81 pieces of actual test data and 791 pieces of augmented test data, 449 people may be classified as belonging to the super-ager group and 361 people may be classified as belonging to the typical-ager group, with respect to a total of 810 pieces of data.

Next, the cognitive ability evaluation method of the present disclosure includes extracting one or more major blood factors associated with the first group for each major cognitive function item of the cognitive function test (660). More specifically, according to an embodiment of the present disclosure, SHAP values of blood factors may be derived for each cognitive function item with respect to the entire test data, and blood factors which influence whether or not being the super-ager may be determined by using the SHAP values. The SHAP is a technique used to describe the prediction of the model. The SHAP value is also referred to as a Shapley value and may measure a degree to which each blood factor feature contributes to the prediction of the model. In particular, the SHAP may describe the prediction by taking into account various combinations while individually calculating the importance of each blood factor feature. That is, the influence of certain blood factors on classification into the super-ager may be quantified by performing SHAP analysis for each cognitive ability item.

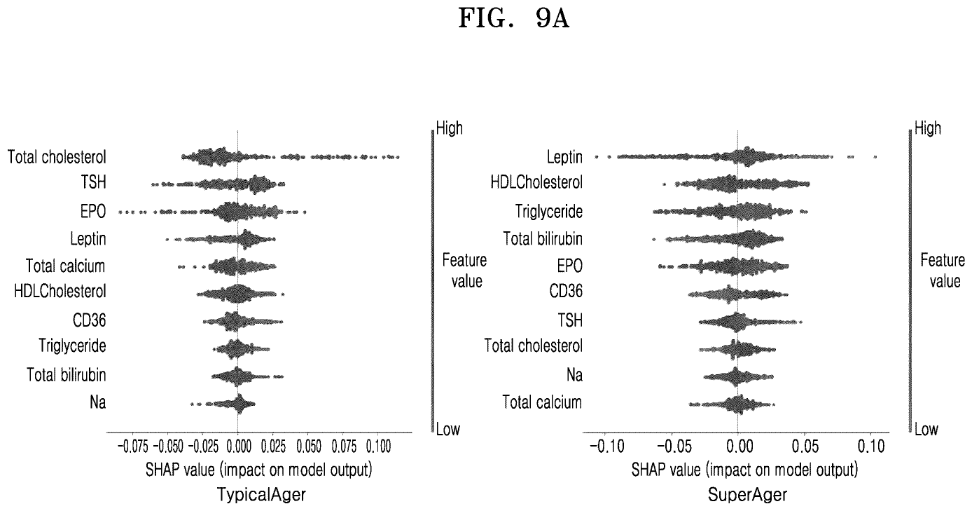

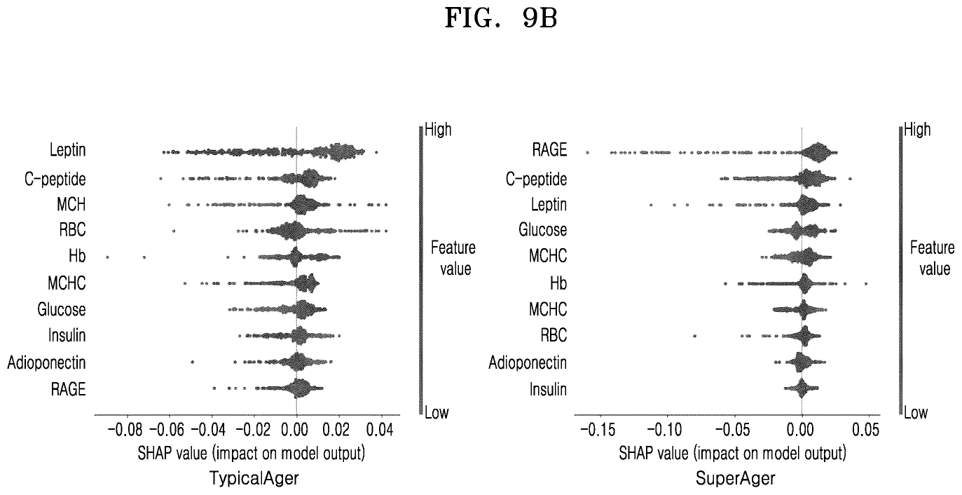

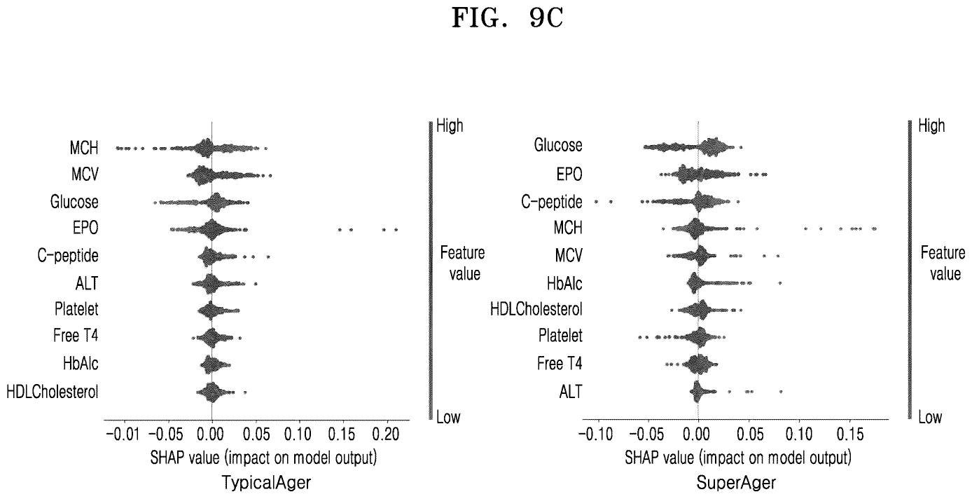

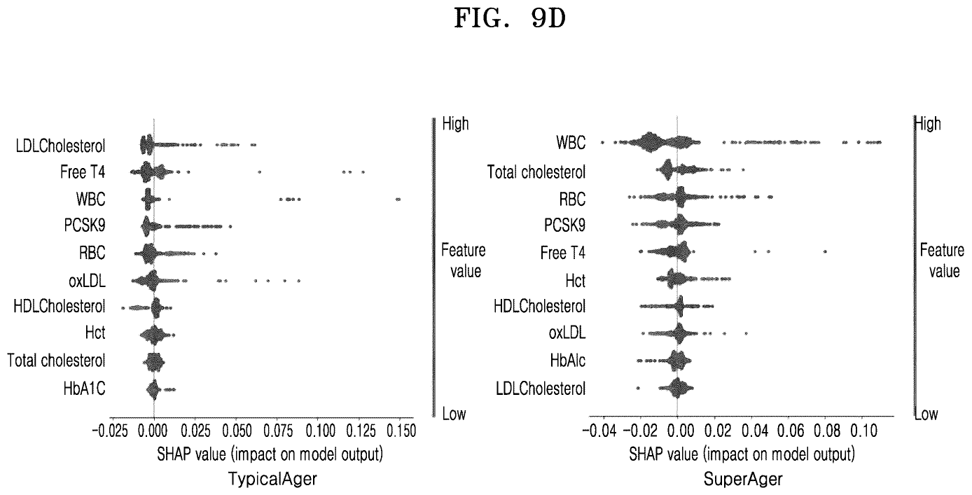

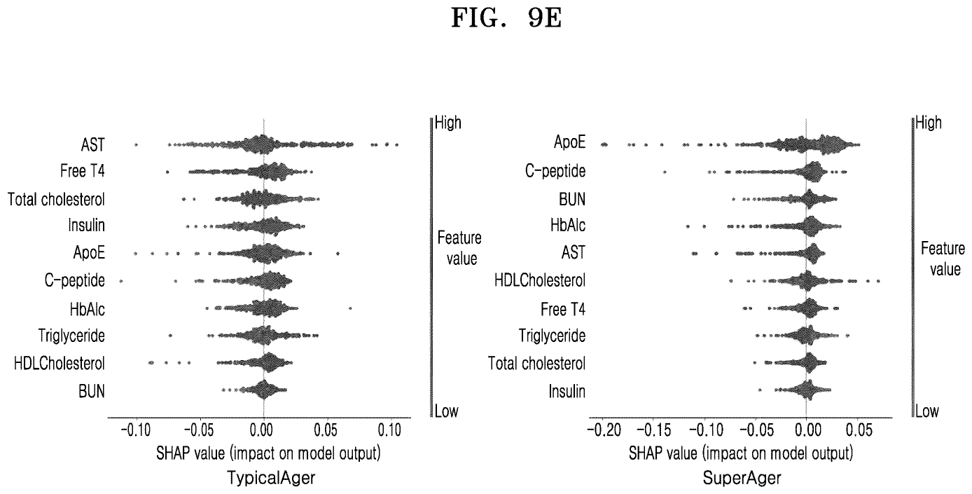

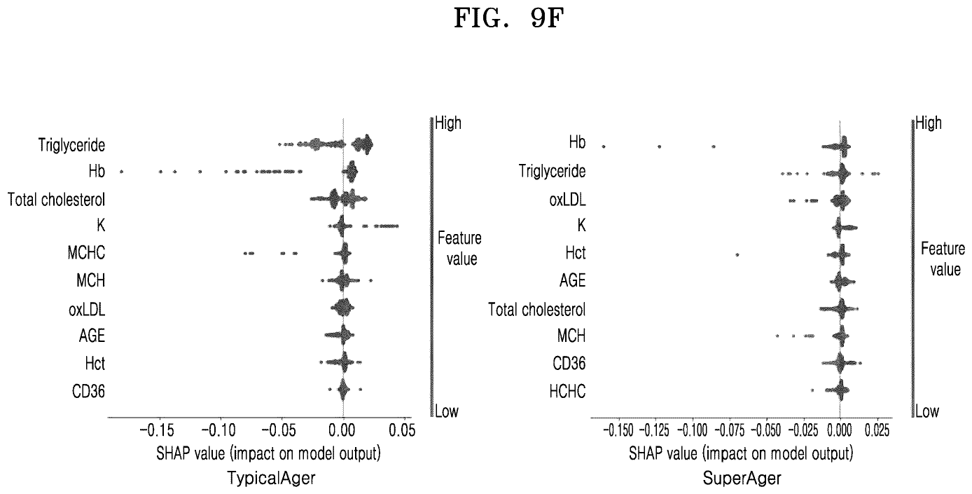

FIGS. 9A to 9E are graphs for describing blood factors associated with the first group determined for each item of the cognitive function test.

Referring to FIG. 9A, when comparing the super-ager group with the typical-ager group, it may be seen that the blood factors in the attention cognitive function item are leptin(↓), HDL(↑), triglyceride(↓), total bilirubin(↑), CD36(↑), and Na(↓). In this case, (↑) means that as the blood factor level is higher, the cognitive function score increases, and (↓) means that as the blood factor level is lower, the cognitive function score increases.

Referring to FIG. 9B, when comparing the super-ager group with the typical-ager group, it may be confirmed that the blood factors in the language cognitive function item are RAGE(↑), Hb(↓), and MCHC(↓).

Referring to FIG. 9C, when comparing the super-ager group with the typical-ager group, it may be confirmed that the blood factors in the verbal memory cognitive function item are Glucose(↓), C-peptide(↓), MCH(↑), HDL(↑), Platelet(↓), and Free T4(↓).

Referring to FIG. 9D, when comparing the super-ager group with the typical-ager group, it may be confirmed that the blood factors in the visual memory cognitive function item are WBC(↓), RBC(↑), and oxLDL(↑).

Referring to FIG. 9E, when comparing the super-ager group with the typical-ager group, it may be confirmed that the blood factors in the visuospatial super-ager cognitive function item are score visuospatial(↑): ApoE(↓), HbA1c(↓), AST(↓), free T4(↑), and triglyceride(↓).

Referring to FIG. 9F, when comparing the super-ager group with the typical-ager group, it may be confirmed that the blood factors in the frontal cognitive function item are CD36(↑). That is, as seen in [Table 1], it may be confirmed that there is no significant difference in frontal cognitive function item between the typical ager and the super-ager.

Next, the evaluation model which evaluates cognitive ability is generated based on the major blood factors determined for each major cognitive function item (670). Because the generating of the evaluation model which evaluates cognitive ability, based on the determined major blood factors, is similar to operation S300 of FIG. 4, a redundant description thereof is omitted. On the other hand, compared to the embodiment of FIG. 4, in the embodiment of FIG. 6, predicting and labeling whether the augmented test data is the super-ager by using the classification algorithm may be further performed, and whether the entire test data is the super-ager may be classified based on the predicted result. Therefore, the major blood factors extracted based on the embodiment of FIG. 6 show a regular distribution in the super-ager group, but show a random distribution in the typical-ager group, and thus, the major blood factors extracted in the embodiment of FIG. 6 may be said to be biomarkers which show the features of the super-ager more effectively.

As described above, in operation S200 of FIG. 4, the processor 120 may extract major blood factors for each item of the cognitive function test by using a preset variable selection algorithm (according to an embodiment, by using selectKBest). On the other hand, according to another embodiment of the present disclosure, one or more feature selections other than selectKBest may be used to select major biomarkers for each cognitive domain. In this case, one or more feature selection methods may be Feature Selection with Recursive Feature Elimination (RFE) combined with Simulated Annealing (SA), Feature Selection (cognitive ability) with RFE, or Feature Selection (cognitive ability) with BOURUTA.

More specifically, the processor 120 according to an embodiment of the present disclosure may extract the top 10 features obtained by performing RFE through RandomForest, Adaboost, or lightGBM classifier depending on whether or not being the super-ager by using Feature Selection (SA) with RFE.

According to a specific embodiment, the processor 120 may perform RFE by using Random Forest to extract the top 10 factors, i.e., EPO, FABP-4, free T4, adiponectin, Vit B12, folic acid, HDLCholesterol, WBC, MCV, and CI. In addition, the processor 120 may perform RFE by using AdaBoost to extract the top 10 features, i.e., HDLCholesterol, FABP-4, EPO, WBC, oxLDL, CD36, RBC, Vit B12, adiponectin, and MCV. In addition, the processor 120 may perform RFE by using lightGBM classifier to extract the top 10 factors, i.e., Vit B12, HDLCholesterol, platelet, total cholesterol, EPO, CI, AST, BUN, CRP, and RDW(CV). In addition, the processor 120 may extract overlapping factors (EPO, VitB12, or HDLCholesterol).

On the other hand, according to the test data of the subjects obtained by the processor 120 in operation S100 of FIG. 4, whether each patient is the super-ager (whether each patient belongs to the first group) is binarily divided, but the individual cognitive function scores are continuous. Therefore, a dataset of a total of 81 people is quantitatively insufficient to detect biomarkers among a total of 56 blood factors through continuous variables.

In this regard, as described above, whether or not being the super-ager is determined through cognitive ability scores according to the attention, language, verbal memory, visual memory, visuospatial, and frontal cognitive domains. Therefore, it is necessary to divide the subjects into the top 25% group among the most important test item scores which determine the cognitive ability score of the relevant cognitive domain and the remaining groups, and to identify the major factors of the top group with good cognitive ability scores for each cognitive domain.

To this end, the processor 120 may perform feature selection (cognitive ability) with RFE. Feature selection (cognitive ability) with RFE may be performed by using RandomForest, Adaboost, and lightGBM classifier depending on whether the group has the highest cognitive ability score for each cognitive domain. The processor 120 may extract overlapping factors (HDLCholesterol, leptin, FABP-3, free T4, glucose, RDW(CV), CD36, ApoE, EPO, Vit B12, RAGE, total cholesterol, oxLDL, FABP-4, PCSK9, and triglyceride) from the top 10 factors obtained therethrough.

In addition, the processor 120 may perform feature selection (cognitive ability) with BORUTA. Feature selection (cognitive ability) with BORUTA may be performed by using R-based BORUTA depending on whether the group has the highest cognitive ability score for each cognitive domain. The processor 120 may extract 10 factors (HDLCholesterol, BUN, ApoE, HbA1c, insulin, EPO, vimentin, FABP-4, free T4, total cholesterol, RAGE, and PCSK9) therethrough.

Next, the processor 120 predicts whether or not being the super-ager with factors extracted by the three methods, i.e., Feature Selection with RFE combined with SA, Feature Selection (cognitive ability) with RFE, and Feature Selection (cognitive ability) with BOURUTA, and determines the accuracy of the prediction result. According to an embodiment, because the accuracy is the highest when predicting super-ager with factors extracted by Feature Selection (cognitive ability) with RFE and Feature Selection (cognitive ability) with BOURUTA, the processor 120 may select a total of 20 factors, i.e., EPO, Vit B12, HDLCholesterol, leptin, FABP-3, Free T4, glucose, RDW(CV), CD36, ApoE, RAGE, total cholesterol, oxLDL, FABP-4, PCSK9, triglyceride, BUN, HbA1c, insulin, and vimentin, as super-ager biomarkers.

On the other hand, according to operation 630 of FIG. 6 described above, augmented test data may be generated so as to increase the accuracy of the evaluation model. In this regard, according to another embodiment of the present disclosure, the augmented test data may be used to verify the evaluation model.

More specifically, the evaluation model was trained by using the generated augmented test data, and the evaluation model was verified by using original data through the large-language model based ‘GReaT’ method. According to a specific embodiment, out of a total of 81 pieces of data, 49 pieces of data were used for augmentation, and 70 pieces of augmented data were obtained. Through this, correlation analysis between the actual data and the augmented data was performed through a total of 20 biomarkers described above, and the difference therebetween was very small.

On the other hand, according to an embodiment of the present disclosure, some of the 20 biomarkers described above may be common to two or more cognitive domains. The biomarkers which are common to the plurality of cognitive domains may be used as indicators to measure cognitive reserve, which may more accurately predict whether or not being the super-ager.

As described above, the cognitive ability evaluation method and device according to the embodiments of the present disclosure may evaluate cognitive ability based on the blood test and may easily obtain cognitive ability evaluation results without a time-consuming cognitive function test or magnetic resonance imaging (MRI) scan, thereby significantly reducing the time and cost required for cognitive ability evaluation.

In addition, the embodiments of the present disclosure may expand accessibility to cognitive ability evaluation. For example, regular checkups for cognitive ability may be conducted by evaluating cognitive ability by using the results of blood tests performed periodically in the elderly people.

In addition, the embodiments of the present disclosure may enable healthy people, not just those with a disease, to easily test and manage their cognitive abilities by evaluating whether the subject has cognitive ability beyond the average cognitive ability of the age group to which the subject belongs, and have the effect of tracking changes in cognitive abilities by providing a relatively objective cognitive ability evaluation method based on a blood test.

The present embodiments may be implemented in the form of a recording medium including computer-executable instructions, such as program modules which are executable by a computer. A computer-readable recording medium may be any available media that are accessible by the computer and may include any volatile and non-volatile media and any removable and non-removable media. In addition, the computer-readable recording medium may include a computer storage medium and a communication medium. The computer storage medium may include any volatile, non-volatile, removable, and non-removable media which are implemented in any method or technology for storage of information, such as computer-readable instructions, data structures, program modules, or other data. The communication medium may typically include computer-readable instructions, data structures, other data of modulated data signals, such as program modules, or other transmission mechanisms, and may include any information delivery medium.

In addition, in the present specification, the term “unit” may refer to a hardware component, such as a processor or a circuit, and/or a software component which is executed by a hardware component, such as a processor.

The present disclosure has been described with reference to the embodiment illustrated in the drawings, but this is only an example. It will be understood by those of ordinary skill in the art that various modifications and variations may be made thereto. Therefore, the true technical protection scope of the present disclosure should be defined by the technical concept of the appended claims.

1. A blood-based cognitive ability evaluation method comprising:
obtaining actual test data including actual blood test results and cognitive function test results of subjects;
determining major cognitive function items of a cognitive function test related to whether the subjects belong to a first group, based on the actual test data;
generating augmented test data, based on the obtained actual test data;
predicting, by using a classification algorithm, whether pieces of individual data of the augmented test data belong to the first group;
classifying the pieces of individual data of the augmented test data and the actual test data into one of the first group and a second group; and
extracting one or more major blood factors associated with the first group for each of the major cognitive function items, based on the classified actual test data and the classified augmented test data.

2. The blood-based cognitive ability evaluation method of claim 1, wherein the determining of the major items of the cognitive function test comprises:
classifying the respective subjects into the first group or the second group;
training a correlation between each item of the cognitive function test and whether the subjects belong to the first group, based on the classification of the subjects into the first or second group and the actual test data; and
determining the major cognitive function items, based on the training.

3. The blood-based cognitive ability evaluation method of claim 1, wherein the first group is a group of super-agers who have cognitive abilities beyond an average cognitive ability of an age group to which the subjects belong, and the second group is a group of typical-agers who are not super-agers.

4. The blood-based cognitive ability evaluation method of claim 1, wherein the generating of the augmented test data comprises generating, by using a preset data generation algorithm, augmented test data including virtual blood test results and cognitive function test results.

5. The blood-based cognitive ability evaluation method of claim 1, wherein the predicting of whether or not belonging to the first group comprises generating primary prediction results by using at least two different classification algorithms for the augmented test data, and generating a final prediction result by stacking the primary prediction results.

6. The blood-based cognitive ability evaluation method of claim 5, wherein the augmented test data is preprocessed by using LabelEncoder and SimpleImputer before being input to the at least two different classification algorithms.

7. The blood-based cognitive ability evaluation method of claim 1, wherein the determined major cognitive function items are verbal memory, visual memory, and visuospatial items.

8. The blood-based cognitive ability evaluation method of claim 1, further comprising generating an evaluation model which evaluates cognitive ability, based on the major blood factors extracted for each of the major cognitive function items.

9. A computer-readable recording medium having recorded thereon a program for causing a computer to perform the method of claim 1.

10. A blood-based cognitive ability evaluation device comprising:
a processor;
a communication unit; and
a memory,
wherein the processor is configured to:
obtain actual test data including actual blood test results and cognitive function test results of subjects;
determine major cognitive function items of a cognitive function test related to whether the subjects belong to a first group, based on the actual test data;
generate augmented test data, based on the obtained actual test data;
predict, by using a classification algorithm, whether pieces of individual data of the augmented test data belong to the first group;
classify the pieces of individual data of the augmented test data and the actual test data into one of the first group and a second group; and
extract one or more major blood factors associated with the first group for each of the major cognitive function items, based on the classified actual test data and the classified augmented test data.
